SEQUENCE LISTING (3 sequences)

SEQ ID NO: 1 - protein, 21 aa, Euphlyctis cyanophlyctis (Skittering frog)
FLNALKNFAKTAGKRLKSLLN

SEQ ID NO: 2 - protein, 21 aa, synthetic construct
NLLSKLRKGATKAFNKLANLF

SEQ ID NO: 3 - protein, 34 aa, Halocynthia papillosa (Red sea-squirt)
GFWKKVGSAAWGGVKAAAKGAAVGGLNALAKHIQ

(19) United States
(12) Patent Application Publication
(10) Pub. No.: US 2025/0368700 A1
(43) Pub. Date: Dec. 4, 2025

(54) NOVEL PEPTIDE FOR TREATING AN INFECTIOUS DISEASE OR CONDITION

(71) Applicant: OMNIX MEDICAL LTD., Jerusalem (IL)

(72) Inventors: Niv BACHNOFF, Modiin (IL); Moshe COHEN-KUTNER, Lotem (IL); Shira MANDEL, Nofim (IL); Noa NUR, Ma'ale Hachamisha (IL)

(21) Appl. No.: 18/875,416

(22) PCT Filed: Jun. 18, 2023
(86) PCT No.: PCT/IL2023/050630

§ 371 Date: Dec. 16, 2024

Related U.S. Application Data
(60) Provisional application No. 63/356,520, filed on Jun. 29, 2022.

(51) Int. Cl. C07K 14/46; A61K 38/00; A61P 31/04

(52) U.S. Cl. CPC C07K 14/463; A61P 31/04; A61K 38/00

(57) ABSTRACT

The invention provides a peptide having antimicrobial activity comprising the amino acid sequence as set forth in SEQ ID No. 1 (FLNALKNFAKTAGKRLKSLLN), wherein at least one, part, or all of the amino acids of SEQ ID No. 1 are in D-configuration. Further provided are methods of using the same for the treatment or prevention of an infectious disease or condition.

### BACKGROUND OF THE INVENTION

Antibiotics are chemical substances having the capacity, in a dilute solution, to kill or inhibit the growth of microorganisms. Antibiotics that are sufficiently nontoxic to the host are used as chemotherapeutic agents to treat infectious diseases of humans, animals, and plants. The term was originally restricted to substances produced by microorganisms but has been extended to include synthetic and semi-synthetic compounds of similar chemical activity.

Extensive and widespread use of antimicrobial drugs led to the emergence of resistant strains of microorganisms. Due to the spread of multi-drug resistant (MDR) and extremely drug-resistant (XDR) bacterial pathogens, even common infections may transform into conditions that are untreatable with the standard of care antibiotics.

These microorganisms are no longer susceptible to currently available antimicrobial drugs. In order to lower or prevent lethal infectious diseases and maintain public health, new antimicrobial agents are required.

The engineering of stable proteins is of great technological and economic importance, since the limited stability of proteins often severely restricts their medical and industrial application. It is therefore an object of the invention to provide novel stable peptide-based antibiotics to control infections caused by MDR and XDR pathogens.

### SUMMARY OF THE INVENTION

In some embodiments, there is provided a peptide having antimicrobial activity comprising the amino acid sequence as set forth in SEQ ID No. 1 (FLNALKNFAKTAGKRLKSLLN), wherein at least one, part or all of the amino acids of SEQ ID No. 1 are in D-configuration.

In some embodiments, the peptide has one or more improved biological properties relative to SEQ ID NO:1, wherein the one or more properties are selected from the group consisting of antimicrobial activity, hemolytic activity, stability, and therapeutic index for a microorganism.

In some embodiments, all of the amino acids of SEQ ID No. 1 are in D-configuration.

In some embodiments, there is provided a peptide having antimicrobial activity comprising an amino acid sequence that has at least 70%, 75%, 80%, 85%, 90%, 95%, 96%, 97%, 98% or 99% sequence identity to the amino acid sequences set forth in SEQ ID No. 1 (FLNALKNFAKTAGKRLKSLLN), wherein at least one, part, or all of the amino acids are in D-configuration. The peptide has one or more improved biological properties relative to SEQ ID NO:1, wherein the one or more properties are selected from the group consisting of antimicrobial activity, hemolytic activity, stability, and therapeutic index for a microorganism. In some embodiments, the peptide of the invention is amidated at the carboxy terminus (C terminus) domain.

In some embodiments, there is provided a therapeutic composition for controlling infection by a microorganism, said composition comprising at least one antimicrobial peptide as described above in a therapeutically effective amount and a pharmaceutically acceptable carrier.

In some embodiments, there is provided a method of treating a subject in need or preventing an infection in a subject caused by a microorganism, wherein the method comprises the step of administering a therapeutically effective amount of a composition comprising the antimicrobial peptide described herein and a pharmaceutically acceptable excipient. In some embodiments, the microorganism is selected from the group consisting of gram-positive bacteria and gram-negative bacteria. In some embodiments, the bacteria is selected from the group consisting of an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, and any combination thereof. The route of administration is intra-arterial, intravenous, intramuscular, intraperitoneal, intraocular, oral, subcutaneous, transdermal, inhalation, intranasal, rectal, vaginal, topical or any combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

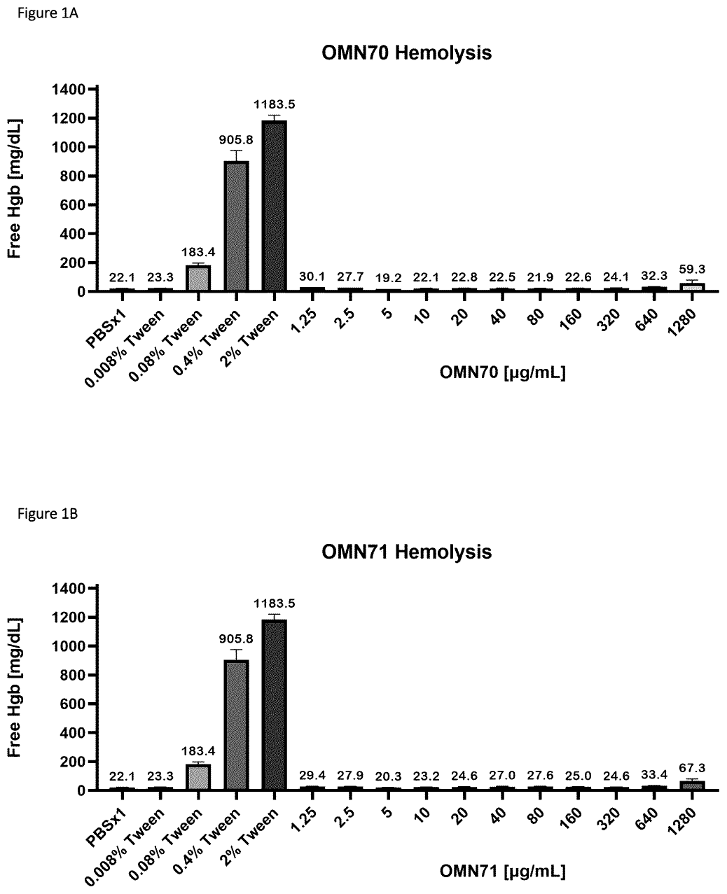

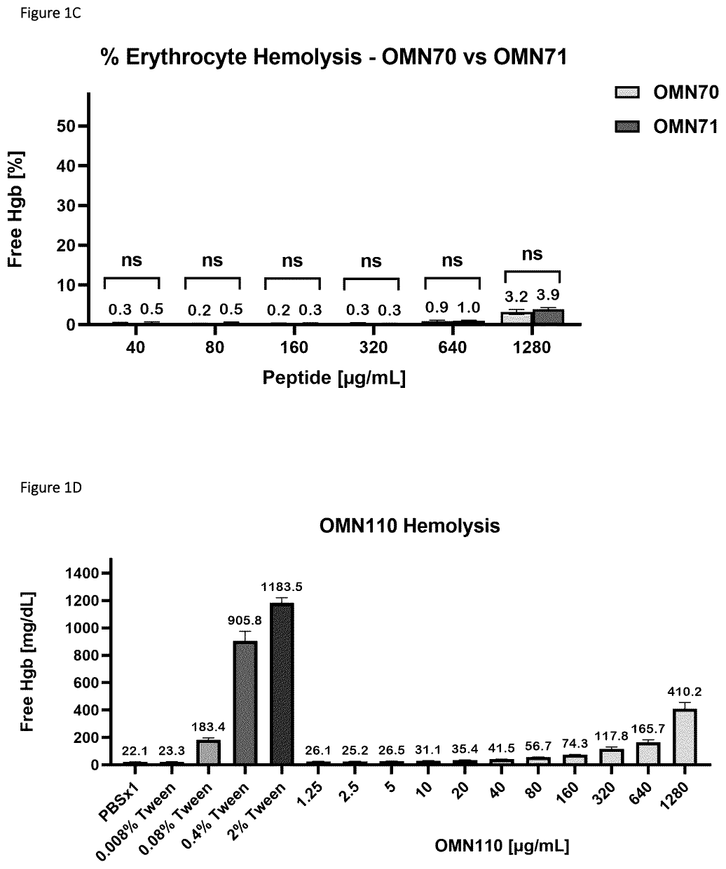

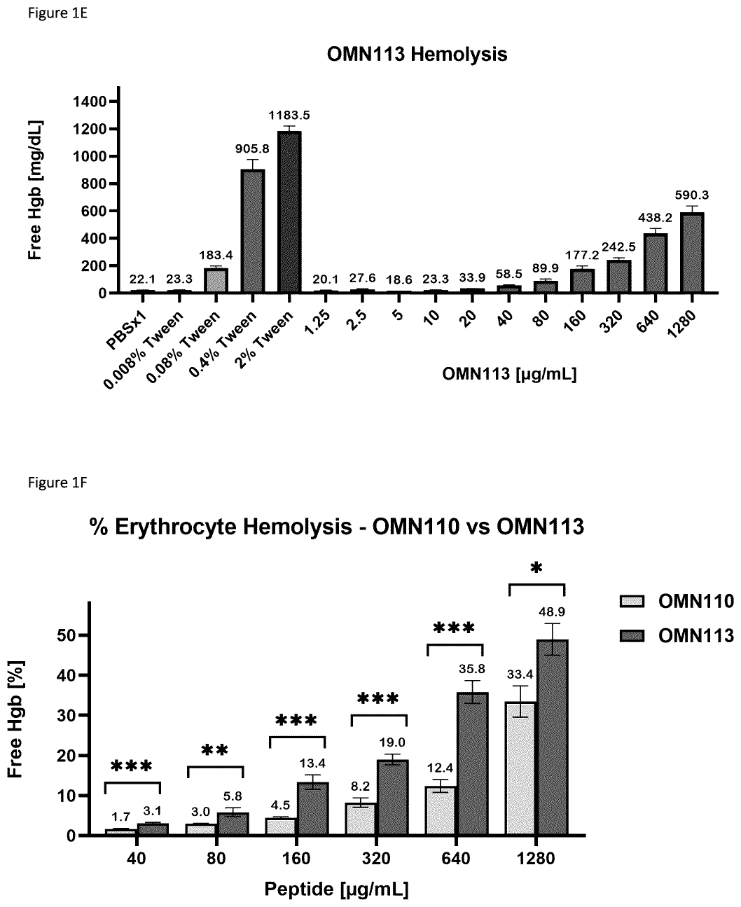

### FIGS. 1A, 1B, 1C, 1D, 1E and 1F: Erythrocyte Hemolysis—Free Hemoglobin Assay

FIGS. 1A, 1B, 1C, 1D, 1E and 1F show results of erythrocyte hemolysis—free hemoglobin Assay. Suspensions of 10% mouse primary erythrocytes were exposed to increasing concentrations of OMN70 and OMN71. The amount of free hemoglobin (Hgb) was assessed via OD at 400 nm and is presented (mg/dL) as an indication of erythrocyte haemolysis. Positive control groups were treated with increasing concentrations of Tween20. Negative control group was treated with PBS (FIGS. 1A and 1B). FIG. 1C demonstrates a comparison of haemolysis levels at peptide concentrations of 40, 80, 160, 320, 640 and 1280 mg/ml, presented as the fraction of free-hemoglobin versus the 2%-Tween20 (%/control), after subtraction of PBSX1 negative control. Not significant (ns): p>0.05; *: p<0.05; **: p<0.01; ***: p<0.001; ****: p<0.0001). FIGS. 1D, 1E and 1F present the same experimental system, peptide treatment and control groups as above conducted with increasing concentrations of OMN110 and OMN113.

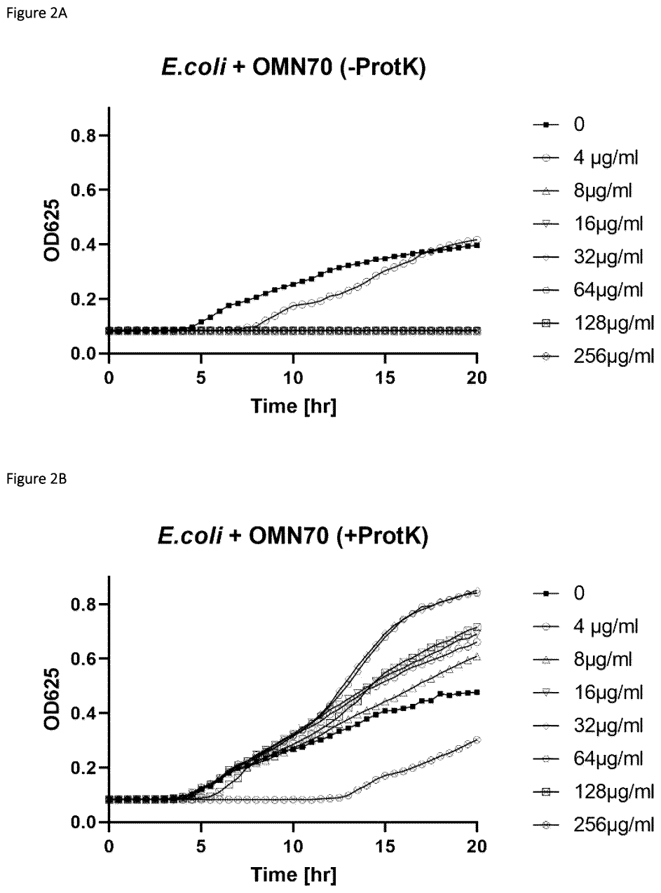

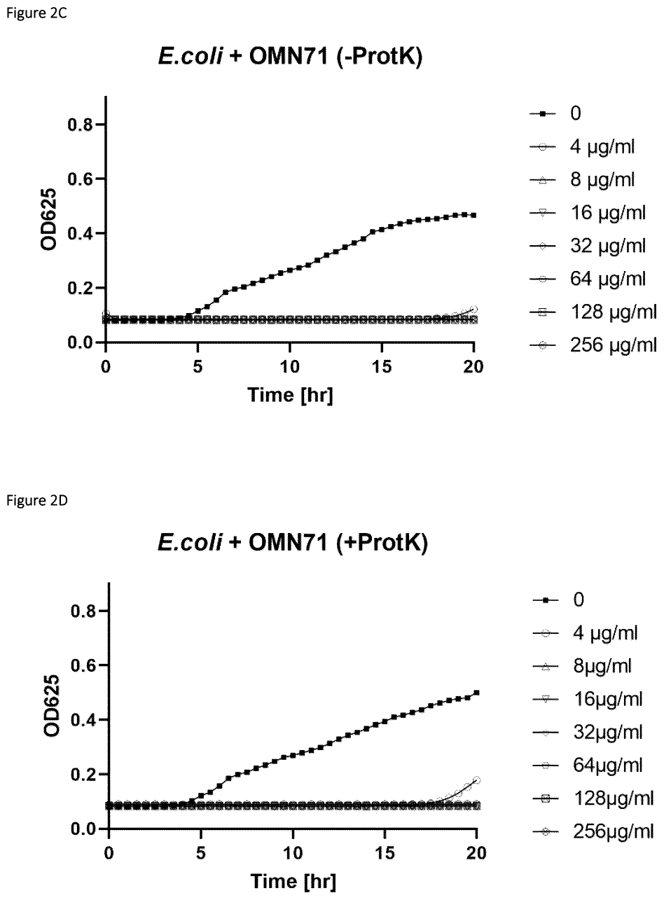

### FIGS. 2A, 2B, 2C and 2D: OMN70/OMN71 Antimicrobial Activity in Presence of Proteinase K

FIGS. 2A, 2B, 2C and 2D show the antimicrobial activity of OMN70 and OMN71 in the presence of proteinase K. FIGS. 2A, 2B, 2C and 2D depict the growth of E. coli bacteria population after incubation w/wo 40 ng of Proteinase K (ProtK). Growth was monitored over 20 hours via absorbance at 625 nm. FIGS. 2A and 2B present and compare the antimicrobial activity of OMN70 on the bacteria population, in increasing concentrations and in the presence or absence of ProtK, as detailed. FIGS. 2C and 2D present and compare the antimicrobial activity of OMN71 on the bacteria population, in increasing concentrations and in the presence or absence of ProtK, as detailed.

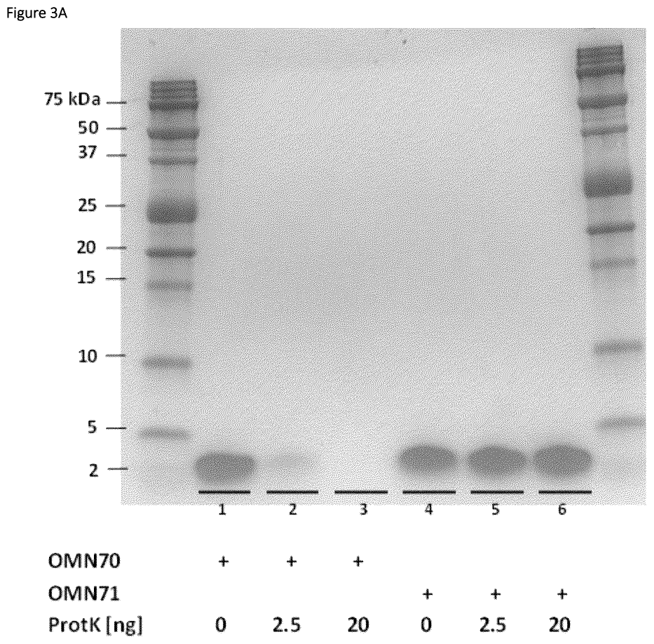

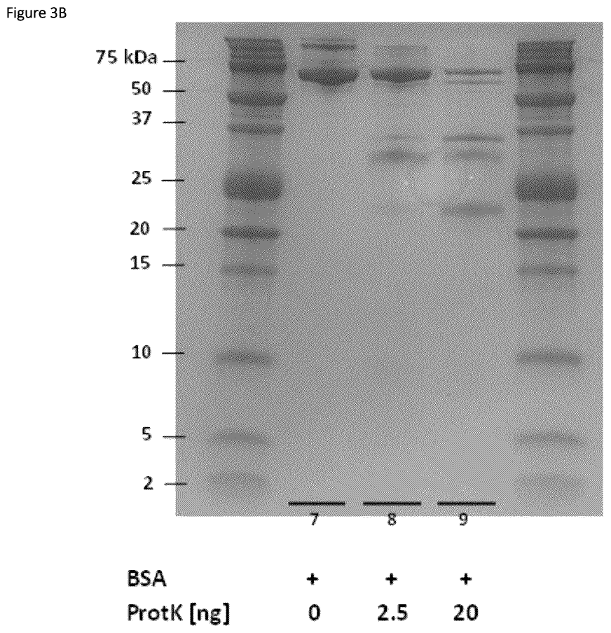

### FIGS. 3A and 3B: Enhanced Stability Against Proteolytic Degradation of OMN71 Vs OMN70 and BSA

FIGS. 3A and 3B depict a Coomassie Blue stained gel detecting the following: FIG. 3A: Lanes 1, 2 and 3: 2.5 mg of OMN70 subjected to 0/2.5/20 ng of Proteinase-K (ProtK), respectively. Lanes 4, 5 and 6: 2.5 mg of OMN71 subjected to 0/2.5/20 ng of ProtK, respectively. FIG. 3B: Lanes 7, 8 and 9: 2.5 mg of Bovine Serum Albumin (BSA) subjected to 0/2.5/20 ng of ProtK, respectively. Proteolysis of OMN70, OMN71 and BSA resulted in the disappearing of the band from the gel.

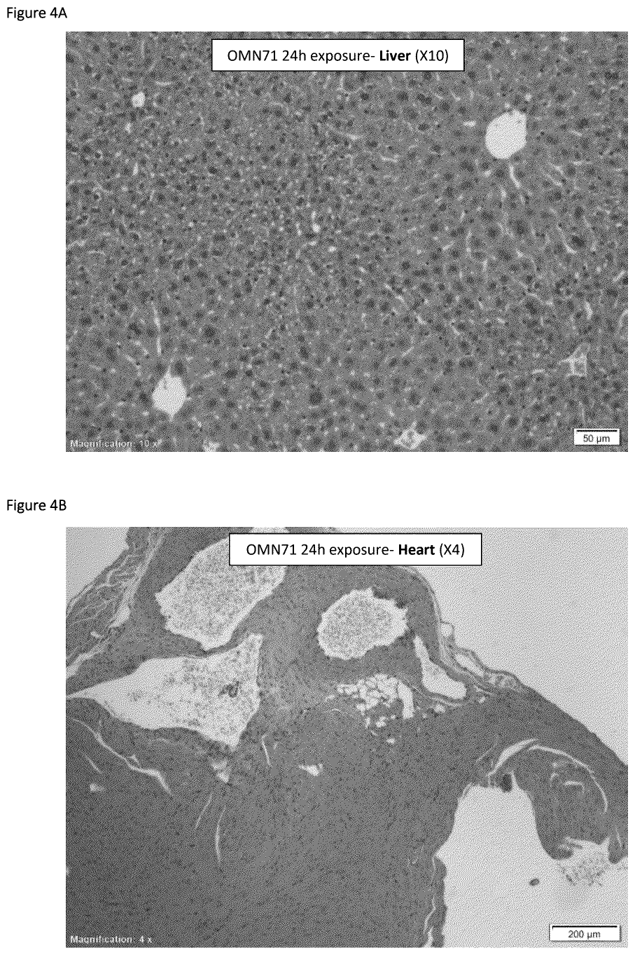

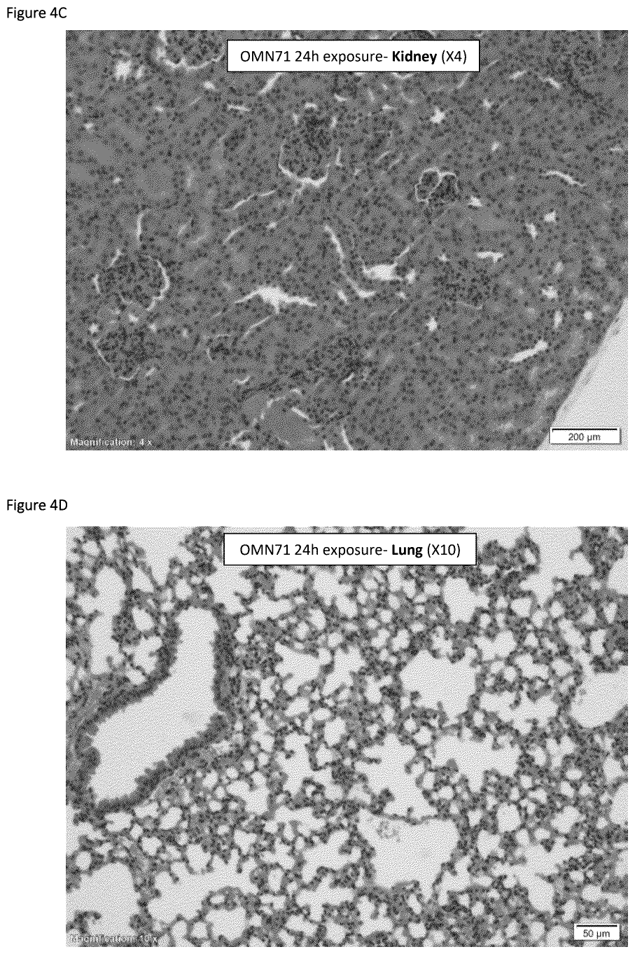

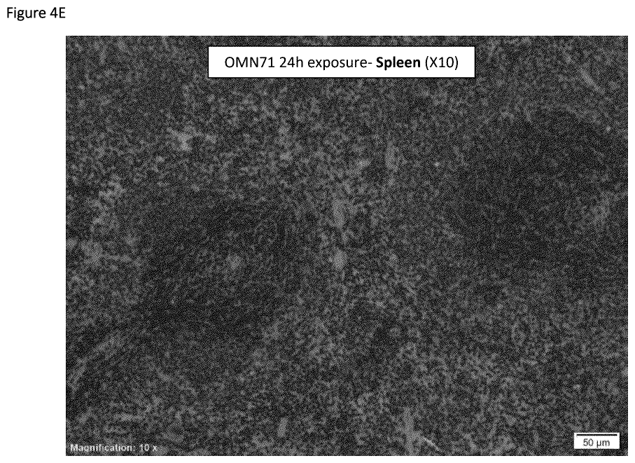

FIGS. 4A, 4B, 4C, 4D and 4E: Histopathological Slides of H&E-Stained Organ Sections from Mice with OMN71 Treatment

CD-1 female mice were intranasally injected (IN) with 100 mg/kg OMN71. At T24h, mice were sacrificed, and spleen, lungs, kidneys, heart and liver were collected for histopathology. Slides of organs were stained with Hematoxylin & Eosin (H&E) and pictures were taken using a microscope equipped with a camera at an objective magnification of ×4 and ×10. FIG. 4A shows the liver histology, FIG. 4B shows the heart histology, FIG. 4C shows the kidney histology, FIG. 4D shows the lung histology and FIG. 4E shows the spleen histology.

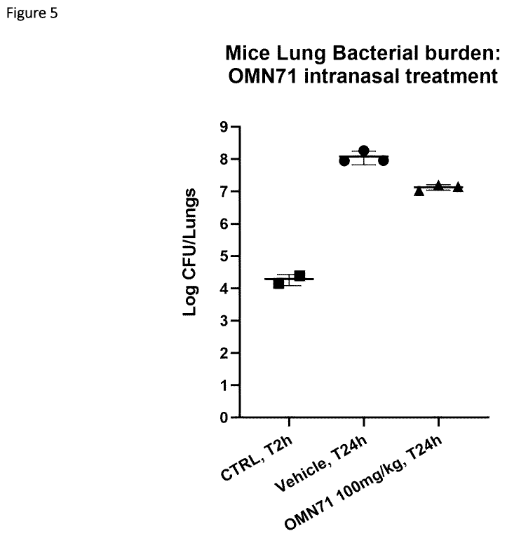

### FIG. 5: Efficacy of OMN71 Intranasal Treatment in the Reduction of Bacterial Burden in a Mouse Lung Infection Model Induced by K. Pneumoniae

Neutropenic CD-1 female mice were intratracheally inoculated with K. pneumoniae strain ATCC 43816 (75×10³ CFU/mouse). Two hours post-inoculation, OMN71-treated group was administered by an intranasal injection of 100 mg/kg and the vehicle group was administered with 0.9% saline solution. Bacterial burden in the lungs was evaluated 24 hours post-infection. Control group of untreated mice n=2 (CTRL ▪), vehicle saline treated mice n=3 (vehicle ●), OMN71-treated group at 100 mg/kg n=3 (OMN71 100 mg/kg ▴).

### DETAILED DESCRIPTION OF THE EMBODIMENTS OF THE INVENTION

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like.

A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances, a disease or condition can be cancer. A subject can be a patient. A subject can be an individual. In some instances, a subject, patient or individual can be used interchangeably.

The terms “treat,” “treating”, “treatment,” “ameliorate” or “ameliorating” and other grammatical equivalents as used herein, can include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition. In some embodiments, the term “treatment” includes the prevention of an infection by bacteria.

The term “preventing” can mean preventing additional symptoms, ameliorating or preventing the underlying metabolic causes of symptoms, and can include prophylaxis.

The terms “effective amount”, “therapeutically effective amount” or “pharmaceutically effective amount” as used herein, can refer to a sufficient amount of a compound being administered which will at least partially ameliorate a symptom of a disease or condition being treated.

The terms “compound”, “agent”, or “therapeutic agent” can be used to refer to a peptide as described herein. In some cases, the terms “additional compound”, “additional agent”, or “additional therapeutic agent” can be used to refer to a peptide as described herein. In some cases, the terms “additional compound”, “additional agent”, or “additional therapeutic agent” can be used to refer to a compound, agent, or therapeutic that may not be a peptide described herein. For example, an additional agent can include an antioxidant, an antibiotic, an antifungal, an antiviral, an antineoplastic, a neoadjuvant, and the like. In some instances, “compound, “agent”, and “therapeutic agent” can be used interchangeably.

The terms “peptide” and “polypeptide” can be used interchangeably to encompass both naturally-occurring and non-naturally occurring proteins, and fragments, mutants, derivatives and analogs thereof. A polypeptide may be monomeric or polymeric. Further, a polypeptide may comprise a few different domains, each of which has one or more distinct activities. For the avoidance of doubt, a “polypeptide” may be any length greater two amino acids. A peptide can comprise an overall charge based on pKa of side chains of component amino acids. In some instances, a peptide can have an overall positive charge. In some instances, a peptide can have an overall negative charge. In some instances, a peptide can have an overall neutral charge. A peptide can furthermore exist as a zwitterion.

A peptide described herein can be useful as an antimicrobial peptide, for example, against bacteria, fungi, yeast, parasites, protozoa and viruses. The term, “antimicrobial peptide” can be used herein to define any peptide that has microbicidal and/or microbistatic activity and encompasses, non-exclusively, any peptide described as having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties.

The term “recombinant” can refer to a biomolecule, e.g., a gene or protein, that (1) can be removed from its naturally occurring environment, (2) can be isolated from all or a portion of a polynucleotide in which the gene may be found in nature, (3) can be operatively linked to a polynucleotide which it may not be linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism can be recombinant, for example, if it is synthesized from an mRNA synthesized from a recombinant gene present in the cell.

In some embodiments, there is provided a peptide having antimicrobial activity comprising the amino acid sequence as set forth in SEQ ID No. 1 (FLNALKNFAKTAGKRLKSLLN), wherein at least one, part, or all of the amino acids of SEQ ID No. 1 are in D-configuration. In some embodiments, the peptide is amidated at the carboxy terminus (C terminus) domain, i.e. FLNALKNFAKTAGKRLKSLLN-NH2 (SEQ ID No. 1).

In some embodiments, the peptide having one or more improved biological properties relative to SEQ ID NO:1, wherein the one or more properties are selected from the group consisting of antimicrobial activity, hemolytic activity, stability, and therapeutic index for a microorganism.

In some embodiments, there is provided a peptide wherein all of the amino acids of SEQ ID No. 1 are in D-configuration.

In some embodiments, there is provided a peptide having antimicrobial activity consisting of the amino acid sequence as set forth in SEQ ID No. 1 (FLNALKNFAKTAGKRLKSLLN), wherein at least one, part, or all of the amino acids of SEQ ID No. 1 are in D-configuration. In some embodiments, the peptide having one or more improved biological properties relative to SEQ ID NO:1, wherein the one or more properties are selected from the group consisting of antimicrobial activity, hemolytic activity, stability, and therapeutic index for a microorganism.

In some embodiments, there is provided a peptide wherein all of the amino acids of SEQ ID No. 1 are in D-configuration.

The term “homology” can refer to a % identity of a polypeptide to a reference polypeptide. As a practical matter, whether any particular polypeptide can be at least 50%, 60%, 70%, 80%, 85%, 90%, 92%, 95%, 96%, 97%, 98%, or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters can be set such that the percentage of identity is calculated over the full length of the reference amino acid sequence and that gaps in homology of up to 5% of the total number of amino acid residues in the reference sequence are allowed.

In some embodiments, there is provided a peptide having antimicrobial activity comprising an amino acid sequence that has at least 70%, 75%, 80%, 85%, 90%, 95%, or 99% sequence identity to the amino acid sequences set forth in SEQ ID No. 1 (FLNALKNFAKTAGKRLKSLLN), wherein at least one, part, or all of the amino acids are in D-configuration. In some embodiments, the peptide having one or more improved biological properties relative to SEQ ID NO:1, wherein the one or more properties are selected from the group consisting of antimicrobial activity, hemolytic activity, stability, and therapeutic index for a microorganism. In some embodiments, all the amino acids of the peptide having antimicrobial activity comprising an amino acid sequence that has at least 70%, 75%, 80%, 85%, 90%, 95%, or 99% sequence identity to the amino acid sequences set forth in SEQ ID No. 1 (FLNALKNFAKTAGKRLKSLLN) are in D-configuration. In some embodiments, the peptide that has at least 70%, 75%, 80%, 85%, 90%, 95%, or 99% sequence identity to the amino acid sequences set forth in SEQ ID No. 1 (FLNALKNFAKTAGKRLKSLLN), may comprise according to some embodiments, substitution, conservative amino acid substitutions, conservatively modified sequence variants, deletion, and/or insertion at one or more position.

In some embodiments, the peptide of the invention is amidated at the carboxy terminus (C terminus) domain.

In some embodiments, there is provided a peptide having antimicrobial activity consisting of an amino acid sequence that has at least 70%, 75%, 80%, 85%, 90%, 95% or 99% sequence identity to the amino acid sequences set forth in SEQ ID No. 1 (FLNALKNFAKTAGKRLKSLLN-NH2), wherein at least one, part, or all of the amino acids are in D-configuration. In some embodiments, the peptide having one or more improved biological properties relative to SEQ ID NO:1, wherein the one or more properties are selected from the group consisting of antimicrobial activity, hemolytic activity, stability, and therapeutic index for a microorganism. In some embodiments, all of the amino acids of the peptide having antimicrobial activity comprising an amino acid sequence that has at least 70%, 75%, 80%, 85%, 90%, 95%, or 99% sequence identity to the amino acid sequences set forth in SEQ ID No. 1 (FLNALKNFAKTAGKRLKSLLN-NH2) are in D-configuration. In some embodiments, the peptide that has at least 70%, 75%, 80%, 85%, 90%, 95%, or 99% sequence identity to the amino acid sequences set forth in SEQ ID No. 1 (FLNALKNFAKTAGKRLKSLLN-NH2), may comprise according to some embodiments, substitution, conservative amino acid substitutions, conservatively modified sequence variants, deletion, and/or insertion at one or more position.

### Synthesis of Antimicrobial Peptides

The peptides described herein can be ordered from a commercial source or partially or fully synthesized using methods well-known in the art (e.g., chemical and/or biotechnological methods). In certain embodiments, the peptides are synthesized according to solid-phase peptide synthesis protocols that are well-known in the art. In another embodiment, the peptide is synthesized on the solid support according to the well-known Fmoc protocol, cleaved from the support with trifluoroacetic acid, and purified by chromatography according to methods known to persons skilled in the art. In other embodiments, the peptide is synthesized utilizing the methods of biotechnology that are well known to persons skilled in the art. In one embodiment, a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression, and the peptide is then isolated from the host organism or the growth medium, e.g., by affinity purification.

The peptides can be also prepared by using recombinant expression systems. Generally, this involves inserting the nucleic acid molecule into an expression system to which the molecule is heterologous (i.e., not normally present). One or more desired nucleic acid molecules encoding a peptide of the disclosure may be inserted into the vector. When multiple nucleic acid molecules are inserted, the multiple nucleic acid molecules may encode the same or different peptides. The heterologous nucleic acid molecule is inserted into the expression system or vector in proper sense (5′.fwdarw.3′) orientation relative to the promoter and any other 5′ regulatory molecules, and correct reading frame.

Purified peptides may be obtained by several methods. The peptide is preferably produced in purified form (preferably at least about 80% or 85% pure, more preferably at least about 90% or 95% pure) by conventional techniques. Depending on whether the recombinant host cell is made to secrete the peptide into a growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

### Antimicrobial Compositions and Formulations

Compositions and formulations that include any one or more of the peptides as disclosed herein are also provided. In one embodiment, the composition includes any one or more of the peptides and a possible pharmaceutically acceptable excipient.

“Pharmaceutically acceptable carriers or excipients” refers to any diluents, excipients, or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, mono-, di-, and triglycerides, fatty acids, fatty alcohols, triglycerides, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, wool fat, sugars such as lactose, mannitol, sucrose, natural polymers (chitosan, dextran, hyaluronic acid, gelatin, collagen, alginate), synthetic polymers and copolymers (Polylactic acid, polylactic-co-glycolic acid and Poly-l-Lactic Acid). Suitable pharmaceutical carriers are described in Remington's Pharmaceutical Sciences, Mack Publishing Company, a standard reference text in this field. They are preferably selected with respect to the intended form of administration, that is, oral tablets, capsules, powders, suspensions, elixirs, syrups, and the like, and consistent with conventional pharmaceutical practices.

The pharmaceutical compositions of the disclosure can be manufactured by methods well-known in the art such as conventional milling, granulating, mixing, dissolving, encapsulating, lyophilizing, or emulsifying processes, among others. Compositions may be produced in various forms, including granules, precipitates, or particulates, powders, including freeze-dried, rotary dried or spray-dried powders, amorphous powders, injections, emulsions, elixirs, suspensions or solutions. Formulations may optionally contain fillers, bulking agents, stabilizers, pH modifiers, surfactants, plasticizers, binders, bioavailability modifiers, pore formers, preservatives and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral, local or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides, phospholipids. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this disclosure are formulated for pharmaceutical administration to a mammal, preferably a human being. Such pharmaceutical compositions of the disclosure may be administered in a variety of ways.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers. In addition, the compound may be formulated for inhaled administration, by dry powder, solution or suspension using an inhalation device.

In addition to dosage forms described above, pharmaceutically acceptable excipients and carriers and dosage forms are generally known to those skilled in the art and are included in the disclosure. It should be understood that a specific dosage and treatment regimen for any particular patient will depend upon a variety of factors, including the activity of the specific peptide employed, the age, body weight, general health, sex and diet, renal and hepatic function of the patient, and the time of administration, rate of excretion, drug combination, judgment of the treating physician or veterinarian and severity of the particular disease being treated.

The compositions of the present invention may be formulated as a unit dosage form. In such form, the preparation is subdivided into unit doses containing appropriate quantities of the active ingredients such as for a single administration. The unit dosage form can be a packaged preparation, the package containing discrete quantities of preparation, for example, an ampule, a capsule, a reservoir, a blister, a dispenser, an adhesive bandage, a non-adhesive bandage, a wipe, a baby wipe, a gauze, a pad and a sanitary pad. Inhalation systems such as dry powder inhalers (DPI), metered dosed inhalers, nebulizers, soft mist inhalers or nasal spray may be used in order to deliver the formulation to the lungs. In some embodiments, the composition can further include a secondary agent, such as, an anti-inflammatory agent, an antimicrobial agent, an antifungal agent, an expectorant agent, a bronchodilator, mucolytic, a cystic fibrosis transmembrane conductance regulator (CFTR) modulator, digestive agent, vitamin, biofilm destruction agents, mucus diluting agents and the like. Non-limiting examples of such antimicrobial agents include imipenem, ceftazidime, colistin, chloroquine, artemisinin, vancomycin and daptomycin.

In some embodiments of the invention, the antimicrobial agent is an antibiotic drug. In some embodiments, the antibiotic drug is a beta-lactam antibiotic selected from the group consisting of penicillins, cephalosporins, cephamycins, carbapenems, ceftazidime, cefotaxime, ceftriaxone, cefpodoxime, and aztreonam.

In some embodiments of the invention, the antibiotic drug can be selected from one or more of the following groups Aminoglycoside, Ansamycin, Glycopeptide, Lincosamide, Macrolide, Monobactam, Nitrofuran, Oxazolidinone, Quinolone/Fluoroquinolone, Sulfonamide, Polymyxin and Tetracycline.

Typically, the composition will contain from about 0.01 to 99 percent of the active ingredient. In some embodiments, the composition will contain from about 20 to 75 percent of an active ingredient and will further contain adjuvants, carriers and/or excipients. Determination of optimal ranges of effective amounts of the active ingredient is within the skill of the art. In some embodiments, the pharmaceutical composition may comprise about 0.01 to about 100 mg/kg body-weight of the peptide. In some embodiments, the pharmaceutical composition may comprise about 0.5 to about 100 mg/kg body-weight of the peptide. In some embodiments, the pharmaceutical composition may comprise about 100 to about 500 mg/kg body-weight of the peptide. In some embodiments, the pharmaceutical composition may comprise about 100 to about 300 mg/kg body-weight of the peptide. Treatment regimens for the administration of the peptide of the present invention can also be determined readily by those with ordinary skills in art. That is, the frequency of administration and size of the dose can be established by routine optimization.

### Therapeutic Methods

Methods of using the peptides, compositions and formulations of the present disclosure are also described. In one embodiment, the methods are for preventing or treating an infection of a microorganism. The microorganism can be a bacterium, such as a Gram-negative bacterium or a Gram-positive bacterium, a fungus, or a parasite.

The peptides, compositions and formulations are also useful for treating a disease or condition associated with an infection, such as wound abscess, catheter biofilm, pneumonia, and bacteremia. In some embodiments, the disease or the condition is blood-stream infection, surgical-site infections, respiratory system infections, gastrointestinal infections, urinary tract infections and/or soft tissue infections.

In some embodiments, the treatment methods further include administration, concurrently or sequentially, of a second secondary antimicrobial agent as described above. Non-limiting examples of such agents include imipenem, ceftazidime, colistin, chloroquine, artemisinin, vancomycin and daptomycin.

The peptides, compositions and formulations of the disclosure may be administered to the systemic circulation via parental administration. The term “parenteral” as used herein includes subcutaneous, intravenous, intramuscular, intra-articular, intra-synovial, intrasternal, intrathecal, intrahepatic, intralesional and intracranial injection or infusion techniques. However, in cases where the infection is local (e.g., on the skin), the composition may be administered locally, such as topically or by inhalation.

In an embodiment, a peptide of the invention is integrated in a larger peptide or protein. In an embodiment, a peptide of the invention is covalently or non-covalently associated with another composition. In a particular embodiment, said another composition is a polymer.

The peptides disclosed have antimicrobial activity against a wide range of microorganisms including gram-positive and gram-negative bacteria. Detailed description of the microorganisms belonging to gram-positive and gram-negative bacteria can be found in Medical Microbiology (1991), 3.sup.rd edition, edited by Samuel Baron, Churchill Livingstone, N.Y. Examples of potentially susceptible bacteria include, but are not limited to, Escherichia coli, Salmonella typhimurium, Pseudomonas aeruginosa, Staphylococcus aureus, Staphylococcus epidermidis, Bacillus subtilis, Enterococcus faecalis, Corynebacterium xerosis, and Bacillus anthracis. The antimicrobial activities of the inventive peptides have been demonstrated herein against the fore-mentioned gram-positive and gram-negative bacteria because it is well known in the art that these bacteria are considered as model organisms for either gram-negative or gram-positive bacteria and thus any biological activity demonstrated against these model organisms are accepted as an indication of that demonstrated activity against the entire family of gram-negative or gram-positive bacteria.

According to some embodiment of the invention, there is provided a method of overcoming inherent or acquired resistance of a microorganism to an antibiotic agent, comprising: contacting the microorganism to the peptide of the invention as described herein. The microorganism is, in some embodiments, Escherichia coli, Klebsiella Pneumoniae, Pseudomonas aeruginosa, Salmonella serotype Typhi, Acinetobacter baumannii, a member of Enterobacteriaceae spp., Pseudomonas spp. Salmonella spp., Acinetobacter spp. or any combination thereof.

As used herein “inherent resistance” of a microorganism to an antibiotic agent refers to a natural resistance to the action of the agent even in the absence of prior exposure to the agent. (R. C. Moellering Jr., Principles of Anti-infective Therapy; In: Principles and Practice of Infectious Diseases, 4.sup.th Edition, Eds.; G. L. Mandell, J. E. Bennett, R. Dolin. Churchill Livingstone, New York USA, 1995, page 200).

As used herein, “acquired resistance” of a microorganism to an antibiotic agent refers to a resistance that is not inhibited by the normal achievable serum concentrations of a recommended antibiotic agent based on the recommended dosage. (NCCLS guidelines).

As used herein, “tolerance” of a microorganism to an antibiotic agent refers to when there is microstatic, rather than microcidal effect of the agent. Tolerance is measured by an MBC:MIC ratio greater than or equal to 32. (Textbook of Diagnostic Microbiology, Eds., C. R. Mahon and G. Manuselis, W.B. Saunders Co., Toronto Canada, 1995, page 92).

As noted above, this invention provides methods of treating infections caused by a microorganism, methods of killing a microorganism, and methods of enhancing the activity of an antibiotic agent. In particular, these methods are especially applicable when a microorganism is resistant to an antibiotic agent, by a mechanism, such as tolerance, inherent resistance, or acquired resistance. In this invention, infections are treated by administering a therapeutically effective dose of a cationic peptide alone or in combination with an antibiotic agent to a patient with an infection. Similarly, the combination can be contacted with a microorganism to effect killing.

In some embodiments, the following conditions/diseases may be treated:

Conditions or diseases related to E. coli strains: urinary tract infection (UTI); enteric infection; invasive infection; prostatitis and pelvic inflammatory disease (PID); intestinal infection; hemolytic-uremic syndrome; hepatobiliary, peritoneal, cutaneous, and pulmonary infections; extraintestinal infection if normal intestinal anatomic barriers are disrupted (eg, by ischemia, inflammatory bowel disease, colonic diverticulitis, or trauma); bacteremia; meningitis and the like.

Conditions or diseases related to Pseudomonas strains: HIV-infected patients; cystic fibrosis; sepsis; skin and soft-tissue infections (burns, Deep puncture wounds, osteomyelitis, folliculitis; Acute external otitis (swimmer's ear); malignant external otitis; Ecthyma gangrenosum); respiratory tract infections (ventilator-associated pneumonia, sinusitis, bronchitis); nosocomial urinary tract infection; corneal ulceration; acute bacterial endocarditis; bacteremia.

Conditions or diseases related to Klebsiella strains: bacteremia, surgical site infections, intravascular catheter infections, and respiratory or urinary tract infections that manifest as pneumonia, cystitis, or pyelonephritis and that may progress to lung abscess, empyema, bacteremia, and sepsis.

Conditions or diseases related to Staphylococcus strains: toxic shock syndrome; scalded skin syndrome; bacteremia; skin infections (impetigo, cellulitis, furuncles and carbuncles, necrotizing skin infections); staphylococcal neonatal infections; pneumonia; endocarditis; osteomyelitis; staphylococcal infectious arthritis; surgical incisions, open wounds, or burns.

Conditions or diseases related to Enterococcus strains: urinary tract infections; endocarditis; intra-abdominal and pelvic infections; skin, soft tissue, and wound infections.

In some embodiments of the invention, there is provided a method of disinfecting a wound comprising contacting the wound with the peptide or the pharmaceutical composition of the invention. The wound may be in some embodiments, a blister wound, a soft tissue wound, a cutaneous abscess, a surgical wound, a sutured laceration, a contaminated laceration, a burn wound, a decubitus ulcer, a stasis ulcer, a leg ulcer, a foot ulcer, a venous ulcer, a diabetic ulcer, an ischemic ulcer, a pressure ulcer, an oral infection, a periodontal disease, a partial thickness burn, or a full thickness burn.

In some embodiments, there is provided a therapeutic composition for controlling infection by a microorganism, the composition comprising at least one antimicrobial peptide as described herein in a therapeutically effective amount and a pharmaceutically acceptable carrier.

In some embodiments, there is provided a method of treating a subject in need or preventing an infection in a subject caused by a microorganism, wherein the method comprises the step of administering a therapeutically effective amount of a composition comprising the antimicrobial peptide as described herein and a pharmaceutically acceptable carrier. In some embodiments, the microorganism is selected from the group consisting of gram-positive bacteria and gram-negative bacteria.

The bacteria, in some embodiments, is selected from the group consisting of an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, and any combination thereof.

Depending on the severity of the condition (e.g., the area, depth and degree of the infection) and the responsiveness of the subject to treatment, dosing can be of a single or a plurality of administrations, with the course of treatment lasting from several days to several weeks, several months or several years, or until a cure is effected or diminution of the infection is achieved. Alternatively, the compositions are administered in order to prevent the occurrence of an infection in a subject at risk of developing an infection (e.g. a subject suffering from a chronic inflammatory disease). The compositions may be administered for prolonged periods of time (e.g. several days, several weeks, several months or several years) to prevent occurrence of an infection.

According to an embodiment of the present invention, the compositions of the present invention are administered at least once a day. According to another embodiment, the compositions are administered twice a day, three times a day or more.

According to an embodiment of the present invention, administering is effected chronically.

According to another embodiment, administering is effected for at least about 10 days, 12 days, 14 days, 16 days, 18 days, 21 days, 24 days, 27 days, 30 days, 60 days, 90 days or more.

The amount of a composition to be administered will, of course, be dependent on the subject being treated, the severity of the affliction, the manner of administration, the judgment of the prescribing physician, etc.

### EXAMPLES

The following peptides were synthesized and used in the examples that follow:

TABLE 1A

| | OMN70 | | |
|---|---|---|---|
| | (Cyanophlyctin) | OMN71 | OMN72 |
| Source | Euphlyctis | Synthetic | Synthetic |
| | cyanophlyctis | | |
| | (Skittering frog) | | |
| Sequence | FLNALKNFAKTAGKR | FLNALKNFAKTAGKRL | NLLSKLRKGATKAFNK |
| | LKSLLN-NH₂ | KSLLN-NH₂ | LANLF-NH₂ |
| | (SEQ ID No. 1) | (SEQ ID No. 1) | (SEQ ID No. 2) |
| Modification | Amidated C- | All-D amino acids. | All-D amino acids |
| | terminus | Amidated C-terminus | reverse order. |
| | | | Amidated C-terminus |
| No. AA | 21 | 21 | 21 |
| Molecular | 2348 Da | 2346.87 Da | 2346.87 Da |
| Mass | | | |
| Format | Lyophilized acetate | Lyophilized acetate | Lyophilized acetate |
| | salt | salt | salt |
| Purity | >95% | >95% | >95% |

TABLE 1B

| | OMN110 | |
|---|---|---|
| | (Papillosin) | OMN113 |
| Source | Halocynthia | Synthetic |
| | papillosa | |
| | (Red sea-squirt) | |
| Sequence | GFWKKVGSAAWGGV | GFWKKVGSAAWGGV |
| | KAAAKGAAVGGLNAL | KAAAKGAAVGGLNAL |
| | AKHIQ-NH₂ | AKHIQ-NH₂ |
| | (SEQ ID NO. 3) | (SEQ ID NO. 3) |
| Mod. | Amidated | All-D amino acids. |
| | C-terminus | Amidated C-terminus |
| No. AA | 34 | 34 |
| Molecular | 3319.94 Da | 3319.94 Da |
| Mass | | |
| Format | Lyophilized | Lyophilized acetate |
| | acetate salt | salt |
| Purity | >95% | >95% |

### Example 1: Determination of Minimum Inhibitory Concentration (MIC) of the Peptides on Growth Inhibition of Various Bacteria

Method: In order to determine the MIC values of the synthesized peptides, growth and inhibition of growth of various bacteria were monitored after treatment with the peptides of the invention using CLSI (Clinical and Laboratory Standards Institute. PA, USA) guidelines. Several bacteria species, resistant and susceptible were cultured with or without the OMN peptides in increasing concentrations of between 0.25-256 μg/ml for 20-24 hours in Muller Hinton Broth (MHB). The growth of the bacteria was continuously monitored via spectrophotometry at 625 nm. As bacterial growth progresses, OD625 nm values raised. In samples where the growth was inhibited, OD625 nm values remained constant. The lowest concentration that inhibited the bacterial growth was determined as MIC.

The results are shown in Table 2:

TABLE 2
Summary of Peptides Minimal Inhibitory Concentration (MIC)
Values on Sensitive and Resistant Bacteria In-Vitro

MIC values (μg/mL)

| | OMN70 | OMN71 | OMN72 |
|---|---|---|---|
| Escherichia coli | 4 | 4 | 32 |
| ATCC ® 25922 (Sens.) | | | |
| Escherichia coli | 8 | 4 | 32 |
| ATCC ® BAA-2452 (NDM-1) | | | |
| Escherichia coli | 16 | 4 | 32 |
| ATCC ® BAA-3048 (MDR) | | | |
| Pseudomonas aeruginosa | 16 | 16 | 64 |
| ATCC ® 27853 (Sens.) | | | |
| Pseudomonas aeruginosa | 8 | 4 | 16 |
| ATCC ® BAA-2795 (MDR) | | | |
| Pseudomonas aeruginosa | 16 | 16 | 64 |
| ATCC ® BAA-2797 (MDR) | | | |
| Klebsiella pneumoniae | 8 | 8 | 16 |
| ATCC ® 700603 (Sens.) | | | |
| Klebsiella pneumoniae | 16 | 16 | 32 |
| ATCC ® BAA-2146 (NDM-1) | | | |

Results: As can be seen, the antimicrobial activity of OMN71 was identical to that of the native form OMN70 derived from skittering frog. MIC values of OMN71 against all the tested bacteria species were the same as with OMN70 and slightly improved results were shown in Escherichia coli and Pseudomonas aeruginosa. In strike contrast, OMN72 presented an antimicrobial activity 2-4 MIC dilutions weaker than OMN70, the native form.

Conclusions: Taken together, the results suggest that after the alterations made to the new peptides versus the native form, OMN70, the antimicrobial activity is retained in OMN71 only and not in OMN72. Thus, it is impossible to predict the outcome of modifying antimicrobial peptides.

### Example 2: Erythrocyte Hemolysis—Free Hemoglobin Assay

Method: Red Blood Cells (RBCs) suspension was prepared from whole blood extracted from the heart of Hsd:ICR (CD-1) mice. Briefly, blood was collected in a 24 U/mL heparin tube to prevent coagulation. The cells were then washed with Phosphate Buffered Saline (PBS; Biological Industries) and centrifuged at 200 g for 10 minutes at room temperature. This operation was repeated three times, then remaining RBCs were resuspended in PBS to form a 10% RBC solution. PBS or Increasing concentrations of OMN peptides or Tween at 0.008%-2% were added to RBCs. After an incubation of 1 h at 37° C. with shaking at 100 rpm, experiment tubes were centrifuged for 10 min at 200 g at room temperature. 50 μL supernatant was extracted from each tube and tested according to the Hemoglobin Assay Kit (Sigma-Aldrich). Hemoglobin levels were determined via absorbance at 400 nm.

Results: FIGS. 1A, 1B, 1C, 1D, 1E and 1F show results of erythrocyte hemolysis-free hemoglobin Assay. Suspensions of 10% mouse primary erythrocytes were exposed to increasing concentrations of OMN70 and OMN71. The amount of free hemoglobin (Hgb) was assessed via OD at 400 nm and is presented (mg/dL) as an indication of erythrocyte haemolysis. Positive control groups were treated with increasing concentrations of Tween20. Negative control group was treated with PBS (FIGS. 1A and 1B). FIG. 1C demonstrates a comparison of haemolysis levels at peptide concentrations of 40, 80, 160, 320, 640 and 1280 mg/ml, presented as the fraction of free-hemoglobin versus the 2%-Tween20 (%/control), after subtraction of PBSX1 negative control. Not significant (ns): p>0.05; *: p<0.05; **: p<0.01; ***: p<0.001; ****: p<0.0001). FIGS. 1D, 1E and 1F present the same experimental system, peptide treatment and control groups as above conducted with increasing concentrations of OMN110 and OMN113.

FIGS. 1A, 1B, 1C, 1D, 1E and 1F present and compare the hemolytic effect of OMN peptides on mouse erythrocytes as free hemoglobin (Hgb) levels. FIGS. 1A, 1B and 1C show similar levels of haemolysis for OMN70 and OMN71 in all concentrations of the tested peptides, with haemolysis levels of 3.2% and 3.9%, respectively, at 1280 mg/ml, the highest concentration that was tested. It is clear that OMN70, the native form, and OMN71 presented the same level of hemolytic activity. FIGS. 1D, 1E and 1F show an increased hemolytic activity of OMN113 compared to OMN110, which is the native form. At concentrations above 40 mg/ml, it can be easily seen that OMN113 is more hemolytic than OMN110. There is a threefold increase in haemolysis at 160 mg/ml and 640 mg/ml, a twofold increase of haemolysis at 40 mg/ml, 80 mg/ml and 320 mg/ml, and over a third increase in haemolysis at 1280 mg/ml.

Conclusions: The results presented in FIG. 1 clearly point to the fact that altering the peptides in a similar way may lead to opposite outcomes. While the alteration of the amino acids of OMN70 from all-L to all-D to produce OMN71 did not change the hemolytic activity and the outcome is a novel peptide with a safety profile worth developing towards a therapy, the alteration of the amino acids of OMN110 from all-L to all-D to produce OMN113 had the opposite outcome. Yielding a novel peptide that is more hemolytic and thus not a candidate for further development.

### Example 3: OMN70/OMN71 Antimicrobial Activity in Presence of Proteinase K

Methods: The susceptibility of OMN70 and OMN71 to proteolytic degradation was assessed and the effects of stability on activity were determined. Native peptide, OMN70, and engineered peptide OMN71 both at increasing concentrations were incubated with 40 ng of Proteinase-K (ProtK) at 37° C. for two hours. Escherichia coli (ATCC® 25922) at 500,000 CFU/ml were incubated in MHB for 20 hours with either OMN70 or OMN71 pretreated with ProtK. Bacterial survival was determined via absorption at OD625 nm.

Results: FIGS. 2A, 2B, 2C and 2D show antimicrobial activity of OMN70 and OMN71 in the presence of proteinase K. FIGS. 2A, 2B, 2C and 2D depict the growth of E. coli bacteria population after incubation w/wo 40 ng of Proteinase K. Growth monitored over 20 hours via absorbance at 625 nm. FIGS. 2A and 2B present and compare the antimicrobial activity of OMN70 on the bacteria population, in increasing concentrations and in the presence or absence of ProtK, as detailed. FIGS. 2C and 2D present and compare the antimicrobial activity of OMN71 on the bacteria population, in increasing concentrations and in the presence or absence of ProtK, as detailed.

The results in FIGS. 2A, 2B, 2C and 2D depict the antimicrobial activity of the peptides OMN70, the native form, and OMN71, in the presence or absence of ProtK. The results clearly show that OMN70 is not stable in the presence of protease ProtK. The peptide loses its antimicrobial activity completely. In the presence of ProtK, OMN70 is not stable, degraded and is not active even at a high concentration of 256 mg/ml (FIGS. 2A and 2B). In strike contrast, the incubation of OMN71 with the aggressive protease, ProtK, did not result in any degradation. OMN71 remained stable in the presence of ProtK, and retained its antimicrobial activity with identical MIC values of 8 mg/ml on E. coli bacteria regardless of ProtK presence.

Conclusions: OMN71 is completely resistant against proteolytic degradation by ProtK. Accordingly, it is suitable for further development as a therapy and this enhanced stability of OMN71 vs the native form OMN70 constitutes an advantage of the bioengineered peptide over the native form.

### Example 4: Enhanced Stability Against Proteolytic Degradation of OMN71 Vs OMN70 and SA

Methods: Proteinase-K (ProtK) was used to assess the stability of OMN71 versus native OMN70 and Bovine Serum Albumin (BSA). 2.5 μg of each protein were incubated with increasing concentrations of 2.5 and 20 ng of ProtK as specified, for 2 hours at 37° C. Samples were boiled at 100° C. for five minutes and separated on 16.5% polyacrylamide gel. The gel was then stained with Coomassie Blue and excess dye was removed overnight.

Results: FIGS. 3A and 3B depict a Coomassie Blue stained gel detecting the following: FIG. 3A: Lanes 1, 2 and 3: 2.5 mg of OMN70 subjected to 0/2.5/20 ng of Proteinase-K (ProtK), respectively. Lanes 4, 5 and 6: 2.5 mg of OMN71 subjected to 0/2.5/20 ng of ProtK, respectively. FIG. 3B: Lanes 7, 8 and 9: 2.5 mg of Bovine Serum Albumin (BSA) subjected to 0/2.5/20 ng of ProtK, respectively. Proteolysis of OMN70, OMN71 and BSA resulted in the disappearing of the band from the gel.

The results clearly show that 20 ng of ProtK was sufficient to completely degrade OMN70 and BSA (lanes: 3, 9 respectively). As can be seen, OMN71 is protected from ProtK proteolysis and was not degraded (lane: 6). Results also show that ProtK at a low concentration of 2.5 ng was sufficient to partially degrade OMN70 and BSA (lanes: 2, 8 respectively). In lane 2, the OMN70 band was weaker than the untreated sample band in lane 1. In lane 8, fragments of degraded BSA were detected, evidence of partial degradation. OMN71 was not degraded by ProtK at all.

Conclusions: These results prove that OMN71, despite the biochemical engineering that was employed, is more stable than its native form, OMN70. The enhanced stability allows OMN71 to remain active in a whole animal context harboring protease.

### Example 5: Safety of Intra-Nasal (IN) Administration of OMN71 in Mice

Methods: Adverse effects of OMN71 on mice following intra-Nasal (IN) administration, safety was evaluated by tissue histopathology and mortality. 100 mg/kg of OMN71 in a total volume of 50 μl saline (0.9% NaCl) was administered intranasal (IN) to Hsd:ICR (CD-1) mice (20-25 g) in order to assess the presence any toxic effects. Control mice were administered with saline solution (0.9% NaCl) as vehicle. On the last time-point of each experiment (at T24h), mortality was assessed, the mice were sacrificed by IP injection of Ketamine-Xylazine and spleen, lungs, kidneys, heart and liver were collected for histopathology.

Slides of liver, heart, kidneys, lungs, and spleen were stained with Hematoxylin & Eosin (H&E) and were sent for evaluation. The H&E-stained slides were examined and pictures were taken using microscope equipped with a camera at objective magnification of ×4 and ×10. The H&E-stained slides were examined, described, and scored by the study Pathologist, using a semi-quantitative grading scale, of 5-point scale, for the severity of the histopathological changes (Schafer et al., Toxicologic Pathology 2018, 46:256-265): Grade 0—The tissue appears normal, without any changes at all; Grade 1—Minimal pathological findings; Grade 2—Mild pathological findings; Grade 3—Moderate pathological findings; Grade 4—Severe pathological findings.

The results are shown in Table 3:

Table 3: a Semi-Quantitative Evaluation of the Histopathological Findings after OMN71 IN Administration

TABLE 3
A semi-quantitative evaluation of the histopathological
findings after OMN71 IN administration

| | Spleen | Lungs | Kidneys | Heart | Liver |
|---|---|---|---|---|---|
| Vehicle, T24 h | 0 | 0 | 0 | 0 | 0 |
| OMN71 100 mg/kg, T24 h | 0 | 0 | 0 | 0 | 0 |

Results: No mortality was observed at T24h. Furthermore animals were monitored for an additional week with no mortality or any other adverse effects. The histopathology of the liver, heart, kidneys, lungs, and spleen of animals was evaluated and no pathological changes were found in the animals' organs as shown in FIGS. 4A-4E and in Table 3.

Conclusion: At effective concentrations, OMN71 does not present any adverse effects in tissues and does not cause mortality. Histopathology analysis did not reveal any evidence of pathological changes or other adverse effects. The results show that OMN71 up to 100 mg/kg does not exert any toxic or otherwise adverse effects when injected IN into mice and is therefore considered a safe treatment dose in mice.

### Example 6: Efficacy of OMN71 in Intra-Nasal (IN) Administration in the Reduction of the Bacterial Burden in a Mouse Lung Infection Model Induced by K. Pneumoniae

Methods: Female CD-1 mice, weighing between 20-25 g were inoculated with K. pneumoniae bacteria to induce a lung infection. All animals were monitored during the entire period of the study and clinical signs were recorded. Briefly, eight animals were rendered neutropenic by two intraperitoneal injections of cyclophosphamide (Sigma-Aldrich, Italy) at 150 and 100 mg/kg, four and one day before the infection, respectively. On the day of the infection, the bacterial challenge was prepared by suspending a few colonies of K. pneumoniae strain ATCC® 43816 (OMN71 MIC=4 μg/mL) in a sterile physiological 0.9% saline solution to reach the turbidity of a 0.5 McFarland. The mice were anesthetized by IP injection of Ketamine-Xylazine at low dose and intratracheally inoculated with 50 μL of the bacterial suspension (75×103 CFU/mouse). Two hours post-infection (T2h), 100 mg/kg OMN71 in a total volume of 50 μl saline (0.9% NaCl) was administered intranasal. The animals were sacrificed by IP injection of Ketamine-Xylazine at: Group 1—untreated group, start of treatment—CTRL—was sacrificed two hours post-infection (T2h) and also at the end of the study. All groups were sacrificed after 24h post-infection. The lungs were collected, weighed, and homogenized, dilutions of lung homogenate were plated on Nutrient agar plates and incubated overnight to determiner lung bacterial burden via CFU count. The mice were monitored during the entire study and scored for clinical signs of infection where no mortality or morbidity were observed.

Results: As shown in FIG. 5, 100 mg/kg OMN71 showed a bactericidal effect with a bacterial burden reduction of ˜1 log CFU/lungs after 24 hours of treatment when compared to the vehicle-treated group after 24 hours.

Conclusion: OMN71 was shown to be effective in alleviating bacterial burden in the lungs of mice. A single treatment of OMN71 can be beneficial in cases of bacterial lung infection.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1. A peptide having antimicrobial activity comprising the amino acid sequence as set forth in SEQ ID No. 1 (FLNALKNFAKTAGKRLKSLLN), wherein at least one, part, or all of the amino acids of SEQ ID No. 1 are in D-configuration.

2. The peptide of claim 1, wherein the peptide having one or more improved biological properties relative to the naturally occurring peptide having a sequence ID as set forth SEQ ID NO:1, wherein all amino acids are in L-configuration, wherein the one or more properties are selected from the group consisting of antimicrobial activity, hemolytic activity, stability, and therapeutic index for a microorganism.

3. The peptide of claim 1, wherein all of the amino acids of SEQ ID No. 1 are in D-configuration.

4. (canceled)

5. (canceled)

6. The peptide of claim 1, wherein all of the amino acids of SEQ ID No. 1 are in D-configuration.

7. A peptide having antimicrobial activity comprising an amino acid sequence that has at least 70%, 75%, 80%, 85%, 90%, 95% or 99% sequence identity to the amino acid sequences set forth in SEQ ID No. 1 (FLNALKNFAKTAGKRLKSLLN), wherein at least one, part, or all of the amino acids are in D-configuration.

8. (canceled)

9. The peptide of claim 7, wherein all of the amino acids of claim 1 are in D-configuration.

10. A peptide having antimicrobial activity consisting of an amino acid sequence that has at least 70%, 75%, 80%, 85%, 90%, 95% or 99% sequence identity to the amino acid sequences set forth in SEQ ID No. 1 (FLNALKNFAKTAGKRLKSLLN), wherein at least one, part, or all of the amino acids are in D-configuration.

11. (canceled)

12. The peptide of claim 10, wherein all the amino acids are in D-configuration.

13. The peptide claim 1, wherein the peptide is amidated at the carboxy terminus (C terminus) domain.

14. A therapeutic composition for controlling infection by a microorganism, said composition comprising at least one antimicrobial peptide of claim 1 in a therapeutically effective amount and a pharmaceutically acceptable carrier.

15. A method of treating a subject in need or preventing an infection in a subject caused by a microorganism, wherein the method comprises the step of administering a therapeutically effective amount of a composition comprising the antimicrobial peptide claim 1, and a pharmaceutically acceptable carrier.

16. The method of claim 15 wherein the microorganism is selected from the group consisting of gram-positive bacteria and gram-negative bacteria.

17. The method of claim 16, wherein the bacteria is selected from the group consisting of an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, and any combination thereof.

18. The method of claim 15, wherein the administration is intra-arterial, intravenous, intramuscular, oral, subcutaneous, inhalation, mucosal, intranasal, transdermal, intradermal, topical, intramuscular, depot injection, intraocular, intraperitoneal, rectal, vaginal or any combination thereof.
